(19) United States
(12) Patent Application Publication
(10) Pub. No.: US 2012/0308579 A1
(43) Pub. Date: Dec. 6, 2012

(54) METHOD OF MAKING AND USING A COMPOSITION FOR DELIVERING VIRAL IMMUNOGEN IMMUNOGLOBULIN INHIBITOR TO THE NASAL PHARYNGEAL MEMBRANE

(75) Inventor: MONTE B. TOBIN, Venice, FL (US)

(73) Assignee: Hi-Tigg, Inc., Venice, FL (US)

(21) Appl. No.: 13/309,325

(22) Filed: Dec. 1, 2011

Related U.S. Application Data
(60) Provisional application No. 61/418,956, filed on Dec. 2, 2010.

(51) Int. Cl. A61K 39/42; A61P 31/16

(52) U.S. Cl. 424/159.1

(57) ABSTRACT

A method of making and using an immunoglobulin package containing one or more viral inhibitors specifically targeted to receptor factors in viruses is provided. The immunoglobulin package substantially prevents the binding of viral immunogens in respiratory tracts of humans or animals and mechanically prevents a virus from reproducing in the nasal cavity. The immunoglobulins are made by inoculating cows with the immunogen(s), allowing the immune response to develop in the animal, harvesting the plasma containing the immunoglobulins, manufacturing the plasma to create a gel or liquid to swab, spray or mist into the nostril of the host. This method will prevent the spread of disease and give the host time to produce their own immunity to the virus for future protection. The invention may be utilized to substantially reduce or eliminate viruses that decrease the health of humans or animals such as Influenza and other respiratory viruses.

### RELATED APPLICATION

This application claims the benefit of co-pending Provisional Patent Application Serial No. 61/418,956, filed 2 Dec. 2010.

### FIELD OF THE INVENTION

This invention relates to compositions, methods of making compositions and methods for delivering effective amounts of viral inhibitors in the form of plasma immunoglobulins to the nasal pharyngeal membranes.

### BACKGROUND OF THE INVENTION

The flu caused by the influenza virus group is one of the most frequently occurring human illnesses and is responsible for substantial morbidity and economic loss. Viral inhibitors in the form of active bovine immunoglobulins are effective anti-viral agents in vitro and in vivo. Any microorganism which colonizes the nasal pharyngeal region of the respiratory tract of its host must possess the capability of sticking or adhering to the surface of the mucus membranes in order to multiply. Influenza viruses are no exception to the rule. These viruses attach with hemaglutinin or H receptors and are released from cells by neuraminidase or N enzymes. The primary site of attachment for these viruses appears to be in the nasal membrane. The viruses spread from the nasal region into the pharyngeal region and can lead to lower respiratory infection leading to pneumonia and possible death.

It takes a significant amount of time to create traditional vaccines to treat animals and humans. Further, even if a vaccine is available, the vaccine does not protect the host immediately. With new virus strains being developed on a yearly basis it is difficult for vaccine producers to keep up with new viruses. This means that vaccines may not protect against the most recently emerging viruses. It is therefore desirable to provide a product that can be created relatively quickly and would allow for immediate protection upon delivery to the direct area of the nasal membrane by blocking initial attachment in the nasal membrane and providing a mechanical barrier to protect the person until their own immune system can build-up protection.

Influenza viruses have been shown to move from one species to another with ease. Avian Influenza viruses have been known to mutate and attach to swine membranes. These viruses can in turn mutate to infect human mucus membranes. An example would be an Avian H1N1 Influenza virus infecting swine which in turn can infect humans and cause major epidemics.

Webby et al 2000 studied the evolution of Swine H3N2 Influenza viruses in the United States. In 1998, a unique event occurred when severe outbreaks of influenza were observed in four swine herds in the US.

The causative agents were H3N2 influenza viruses with two antigenically distinct re-assortant viruses being isolated. These viruses contained avian-like genes clusters. Analysis in 1999 of the swine H3N2 isolates showed distinct human-like hemagglutinin (HA) molecules. These genes may confer a selective advantage in pigs. Upon acquiring the full complement of re-assortant genes (i.e., swine, avian or both), the virus becomes better adapted to swine and rapidly spread. (Webby, R. J., S. L. Swenson, S. L. Krauss, P. J. Gerrish, S. M. Goyal, and R. G. Webster, Evolution of swine H3N2 Influenza viruses in the United States, J. of Virology 74(18): 8243-8251, 2000.)

Scholtissek et al 2002 studied the cooperation between the hemagglutinin of avian viruses and the matrix protein of human influenza A viruses. New pandemic human influenza A viruses can be created when re-assortment causes the HA gene of the prevailing human strain to be replaced by the allelic gene of an avian influenza A virus such re-assortment occurred in the 1957 and 1986 influenza pandemics. In studies done in infected Madin-Darby canine kidney cells with amantadine, most of the avian HAs efficiently cooperated with the early human A/PR/8/34 (H1N1) virus M gene but not the most recent human isolates A/Nanchang/933/95 (H3N2). These results suggest that the currently prevailing human influenza A viruses might have lost their ability to undergo antigenic shift and are unable to form new pandemic viruses that contain avian HA. This finding is of great interest for pandemic planning in the future. This knowledge can be utilized to formulate immunogens that have cross-reacting sites that are not normally put into current human vaccines. (Scholtissk, C., J. Stech, S. Krauss, and R. G. Webster, Cooperation between the hemagglutinin of avian viruses and the matrix protein of human influenza A viruses, J. Virology, 76(4): 1781-1786, 2002.)

Widjaja et al 2004 studied the ecology and emergence of Influenza A viruses. Influenza A viruses can infect a variety of species, including birds, pigs and humans. Ecological studies of these viruses have established that wild aquatic birds are the primary source of influenza A viruses. These viruses appear to be in evolutionary stasis while residing in asymptomatic aquatic birds, but they rapidly evolve once they cross species barriers and thus cause mild to severe disease in the new hosts. These viruses cause disease in domestic poultry, pigs and more importantly can also cause human pandemics. An important implication of phylogenetic studies is that the ancestral viruses that caused outbreaks in humans provided gene segments for viruses that caused the 1918, 1957 and 1968 pandemics continue to circulate in wild birds with few mutations. Therefore, intensive surveillance of influenza viruses in aquatic birds can provide information about future outbreaks in domestic species and humans. (Widjaja, L., S. L. Krauss, R. J. Webby, T. Xie and R. G. Webster, Matrix gene of influenza A isolated from wild aquatic birds: ecology and emergence of influenza A viruses, J of Virology 78(16): 8771-8779, 2004.)

Macklin, et al. 1998 studied the immunization of pigs using a particle-mediated DNA vaccine to Influenza A Virus and then challenging with a homologous virus. The vaccines did not prevent initial infection or nasal virus shedding but did limit the infection and resulted in early clearance of the virus. This porcine influenza A virus system is a relevant preclinical model for human studies in terms of disease and gene transfer to the epidermis and thus provides a basis for advancing the development of DNA-based vaccines. This information would lead to the conclusion that standard methods of vaccine development using whole or natural shared parts of the virion would make better immunogens than the current DNA-based vaccines. (Macklin, M. D., D. McCabe, M. W. McGregor, V. Neumann, T. Meyer, R. Callan, V. S. Hinshaw and W. F. Swain, Immunization of pigs with a particle-mediated DNA vaccine to influenza A virus protects against challenge with homologous virus, J. of Virology 72(2): 1491-1496, 1998.)

In early 2005, scientists discovered the development of new strains of a highly pathogenic avian influenza H5N1 that caused devastating economic losses in the poultry industry in Asia and Mexico. These strains have been linked to over 64 human deaths. It may be at least a year more before a vaccine can be developed to protect humans. It is therefore desirable to provide a product that alleviates the spread of a virus immediately.

### SUMMARY OF THE INVENTION

The present invention provides a method for the production of a viral inhibitor for administration to humans and animals to substantially prevent the attachment of viral immunogens or haptens in nasal pharyngeal region of humans and animals, which are themselves subject to target illness.

The method preferably includes inoculating cows, with a particular target immunogen(s). The target immunogen(s) with which the cow is inoculated depends upon the anticipated use of the inhibitor. For example, the objective may be to block the attachment of the virus and/or to substantially reduce or eliminate infection. The current strains of Influenza viruses of Group A would make a good target immunogen pool. It should be understood that the more cross-reactivity is present in the immunogens, the better the final immunoglobulin package will be.

After a period of time sufficient to permit the production in the animal of immunoglobulin to the targeted immunogen or immunogens, plasma from the cow is harvested. The total immunoglobulin-containing contents of the plasma are harvested. The immunoglobulins may be used directly as a liquid, dried or mixed with extenders and carriers to form a gel, mist or spray. The liquid immunoglobulin material may be sprayed into the nostrils of the host or placed as a gel on a swab and swabbed into the nostrils of the host. This direct application forms a mechanical barrier to block the attachment of the viruses to the nasal membranes.

The plasma containing the immunoglobulin specific to the targeted immunogen is preferably administered to humans or animals by distributing the immunoglobulin material substantially uniformly into the nasal passages. When blockage of the attachment of the virus is the objective, the immunoglobulin package is mixed in a special formula and made into a spray or gel is supplied to humans or animals prior to exposure or immediately after an outbreak. The substantial prevention of viruses in the nasal pharyngeal region tract of humans or animals will ultimately permit substantial reduction or elimination of the virus from the human or animal. This repression or blocking of virus will permit a significant decrease in shedding and passage of the virus from host to host. In addition, the resulting decrease in shedding should further enhance the prevention of epidemic and pandemics.

The invention is directed particularly to the production of an inhibitor which is specific to virus attachment factors and to the substantial reduction or elimination of respiratory problems caused by these viruses. The invention is described with particular reference to blocking of viruses but it is understood that the invention is not so limited, and is equally applicable to elimination of illnesses or the elimination of shedding caused by the other colony-forming immunogens and haptens. The invention is flexible enough to allow for the development of immunoglobulins directed to more than one specific viral attachment factors (i.e. H1, H3, H5 or N1, N2, N3,etc.) which may then be mixed in a gel or spray for more broad coverage or mechanical blocking of the viruses.

One aspect of the method of the present invention includes inoculating cows, in or about to reach 2-3 years of age, with specific viral H or N producing immunogen, allowing a period of time sufficient to permit the production in the cows of immunoglobulins to the H or N producing immunogen, harvesting plasma from the cow, separating the immunoglobulin-containing contents of said plasma from the cow, and mixing the separate immunoglobulin batches to a form a broad spectrum immunoglobulin package.

The method may include the colony forming immunogen being from the class consisting of H5N1.

The method may include the colony forming immunogen being from the class consisting of H1N1

The method may include the colony forming immunogen being from the class consisting of H3N2

The method may include mixing the separated immunoglobulin-containing contents of said plasma with a material to form a gel.

The method may include the colony forming immunogen being from the class consisting of any Influenza group antigens.

The method may include mixing the separated immunoglobulin-containing contents of said plasma with a material to form a spray.

The method may include mixing the separated immunoglobulin-containing contents of said plasma with a material to form a mist.

The method may include mixing one or more influenza immunogens of either the H or N antigens one or more other shared immunogens such as the M antigen.

The method may include mixing one or more influenza immunogens of either the H or N antigens with one or more other shared immunogens such as the M antigen where they are shared through the Influenza virus group and may be used to type said virus.

The method may include the broad spectrum immunoglobulin package being mixed with other immunoglobulin packages.

The method may include the immunoglobulin packages being mixed with at least one of a variety of carriers such as soy oil, PBS, whey or other proteins to form a spray, mist or gel.

The method may include administering the immunoglobulin packages to at least one human to control the incidence of influenza by preventing the adherence of viral producing immunogens in the upper respiratory tract including the nasal pharyngeal region.

The method may include administering the immunoglobulin packages to at least one a human by swabbing the nostrils of the human with the immunoglobulin packages.

The method may include administering the immunoglobulin packages to at least one a human by spraying the immunoglobulin packages into the nostrils of the human.

The method may include administering the immunoglobulin packages to at least one animal to control the incidence of influenza by preventing the adherence of viral producing immunogens in the upper respiratory tract including the nasal pharyngeal region.

The method may include administering the immunoglobulin packages to at least one animal by swabbing the nostrils of the animal with the immunoglobulin packages.

The method may include administering the immunoglobulin packages to at least one animal by spraying the immunoglobulin packages into the nostrils of the animal.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

The present invention is based on the concept of specifically inhibiting the ability of viruses to adhere in respiratory tracts of humans or animals and thus reduce the ability of the organisms to multiply, colonize and be shed by the hosts. While the viral inhibitors of the present invention may be administered at will to the host, it is preferred for efficient utilization that a carefully determined and managed course of administration during the incubations period after exposure be scheduled and followed. Such a predetermined period will take advantage of the low dose, a longer cumulative effect, and is also easily integrated into current practices which will provide an economically attractive rate of return through reduction in infection rates within the populations.

For the elimination of viruses, the viral inhibitor of the present invention may be administered either immediately or over some substantial period of the time during the day. It is preferred that a carefully determined and managed mid-term period course of administration be followed. The inhibitor may be administered by a spray, a mist or a gel swab one to three times each day until the host develops their own immune protection.

Any virus such as influenza that colonizes the respiratory tract of its host must possess the capability of sticking or adhering to the cell surface in order to multiply and grow. The viruses addressed by this invention are no exceptions to this rule. As other factors must also be considered, specific reagents are required to reduce the number of targeted viruses in the respiratory tract while not interfering with the normal flora of the respiratory tract. The virus inhibitor of this invention strongly interferes with adherence of viruses in a highly specific manner and, on a cumulative basis, thereby preventing the targeted organisms from multiplying, colonizing and shedding.

Through the vehicle of a simple daily spray or gel swab, the product essentially supplies the host with an antibody preparation designed not to cure any disease in the animal, but to dislodge any resident virus in the respiratory tract and to prevent attachment of any newly introduced numbers of that same virus. The virus inhibitor has no direct effect on the host and leaves no undesirable residue in the host. In addition, since the deleterious viruses are prevented from multiplying, they disappear over time through natural degradation from the environment since viruses can not survive without living cells. This will help to eliminate a significant potential source of recontamination. The inhibitor product itself can be classified as a natural material of animal origin and as such can be used in almost any kind of prevention program. As the active ingredients of the inhibitor are completely natural, they will work well with most additives known in the industry.

All mammals and birds provide various types of immediate passive immune response which protect their very young offspring until they acquire the ability to make immunoglobulins for themselves. More specifically called passive protection, this defense mechanism is passed to the young of mammals through the placenta, colostrums, the mother's milk, or through combinations of same. Bovine immunoglobulins are much more stable and resistant to inactivation through digestion than other mammalian immunoglobulins, especially under adverse conditions. The large quantities of immunoglobulins in the plasma are much more exclusively those specific for the immunogens to which the mother has most recently been exposed to and challenged by. These factors result in the plasma of the cow being the most ideal source for large quantities of economically produced, highly specific and stable immunoglobulins. While the invention is illustrated by the use of bovine to produce immunoglobulins, other mammals including sheep, goats, equine, etc. may be used.

It is contemplated that groups of cows are first obtained. The cows are preferably Holsteins, Jersey, Short horn hybrid crosses, Guernsey or other breeds of average size. Preferably the cows are former dairy cows. The animals will then preferably be subjected a suitable period of isolation and acclimatization of about 2 to 4 weeks, after which each group of cows will enter into an inoculation program using specific immunogens to which an immunoglobulin is desired. The immunogens may be a proprietary preparation of immunogens. The immunogens may be obtained from commercial sources such as the American Type Culture Collection (ATCC) or from environmental isolates. The animals may be vaccinated on a schedule predetermined by the amount and timing of final product desired in order to provide a steady continuous production stream of immunoglobulins. The immunogens may be injected intramuscularly, but are preferably injected subcutaneously in the neck region. In approximately four weeks, the average animal will have produced a substantial amount of immunoglobulin in the plasma. The plasma will then be collected using any means known in the art. The collected plasma will contain copious amounts of the desired specific immunoglobulin in a readily usable and stable form. The cows may be reinoculated with the targeted immunogen as needed throughout the period to maintain a high immunoglobulin level.

Batches of plasma from each group of cows are preferably harvested on a weekly basis. The plasma is preferably tested using any means known in the art to determine the immunoglobulin levels. Then the plasma from various groups of cows may be mixed to create an immunoglobulin product that has the desired characteristics. The typical batch is blended with batches from other groups of cows at other average production levels resulting in a production lot with a standardized active ingredient level. The plasma is preferably filtered using any method known in the art to eliminate potential pathogenic microorganisms from the cow and thus reduce potential contamination of product.

The immunoglobulin mixture can be used directly with standard buffers or purified using an extraction buffer to form mixture of immunoglobulins. These extracted preparations can be sterilized using filtration. If desired, the immunoglobulin packages can be mixed with carriers and used as a gel, mist or spray. Dependent on the needs and specifications of the product formulator and the final customer, the final product may include some type of innocuous additive, such as buffer, glycerin, or the like to formulate a nasal spray. If desired, the plasma may be dried using standard commercial methods, such as spray drying using ambient or hot air up to 50° C. and tested to determine overall titer or immunoglobulin level. The immunoglobulins may be used as a liquid alone or on dried extenders such as gel gum or rice hulls or the like as is known in the art. Standard test procedures, including but not limited to ELISA or agglutination, may be used to test the immunoglobulins.

Plasma produced and processed by the above procedures will yield a product sufficiently active and stable to provide protection against virus colonization. This method provides for the first time, an economical, safe, and effective means for controlling viruses in humans or animals. It should be understood that if desired, the collected immunoglobulin package can be passed through a concentrator if the package needs to be more concentrated.

The present invention specifically addresses viral attachment as it relates to respiratory tracts of humans and animals, and to the problem of eliminating viruses from respiratory tracts. However, the concept of preventing viral adherence has great economic potential for a number of diverse food and human safety and production applications. One such field of application is for use in feed and water to target specific undesirable microorganisms. An example of this application would include products to actively inhibit microorganisms in animal feed formulated for swine, cattle, chickens and other poultry. This may prevent or block the spread of the targeted microorganisms from animal to animal or bird to bird. Another such field of application is for rinse ingredients targeted to specific undesirable microorganisms from the environment.

The most successfully colonizing viruses have evolved a number of different types of molecules, referred to as “receptors,” on their surfaces which can very tightly stick to one or more molecules that are part of the host's cell surfaces. These “receptors” attach themselves to their hosts with a lock and key type of fit to very unique chemical structures. The immunoglobulin packages of the present invention may contain inhibitors such as a bovine immunoglobulin of extraordinarily high specific activity which can very tightly bind to coat, cover, and obliterate these “receptors.” In addition to this direct attack, components of the complement system included in most biological fluids, such as blood, mucus, lymph, saliva, tears, and to some extent, intestinal secretions, recognize an immunoglobulin attachment as triggers for their many types of defensive activities.

The invention is further illustrated by the following examples:

### Example 1

### Selection of Cows for Immunization

The strain of bovine used may vary with needs and uses. Any bovine animal may be immunized including dairy cattle, cows, steers or even bulls. Culled dairy cows are preferred because they have been trained to stand in holders for long periods of time. The common strains of bovine are preferred and are usually selected for the concentration of immunoglobulins they can generate and ease of handling. Jersey, Guernsey and Holstein cows of average dairy size usually meet these criteria. The short-horned (polled) animals work the best as to gentle handling. Animals can be selected from culled cows on a farm or at sale barns. All animals must have a clean record of good health. Animals that are older (2-3 years or more) have been found to have the best profile for immunoglobulin patterns. All animals are tested for BVD, Johnes and Mycoplasma. This may be done at certified laboratories using direct counts and PCR testing. Immunoglobulin profiles using the Immunogen ELISA's are done on the individual serum samples. Once the animals meet the initial specifications they are divided into groups. For Example, if the animals have good concentration of immunoglobulins to H1 or N1, they can be placed in the H1N1 group. At least two animals are needed per group but as many cows as needed can be added to the H1N1 group. They are then vaccinated according the schedule given in Example #3. The plasma is then harvested as needed. These animals can be utilized until no longer needed. Depending upon the schedule, the animals may be needed to be boostered on a quarterly basis as needed.

### Example 2

### Preparation of Swine Influenza (H1N1, H3N2) Virus antigens for Model “SI” Immunogens

Stock cultures from ATCC may be used as seed materials or commercial live vaccines of Swine Influenza (H1N1, H3N2)(SI) viruses. The starting materials may then be processed and made into SI immunogens. Individual vials of H1N1 and H3N2 viruses may be obtained from ATCC. Following the directions, the viruses may be propagated in 9-10 day old fertile eggs. The fluid may be collected after 48 hrs and placed into flasks. Flasks may be combined and the material may be harvested using any method known in the art, including but not limited to centrifugation and sterile saline, PBS or culture medium. The material may be diluted to approximately 1×10⁹cfu per ml. Four tenths of percent (0.4%) deoxycholate solution may be added as a 1:1 ration with culture in 0.9% sterile saline (Herzberg et al, 1972) and stirred for approximately 18 hours at room temperature (22° to 24° C.). The material may be centrifuged to remove debris. Supernatant may be used as stock for virus antigen. The dry weight is determined. The product may be diluted in sterile PBS, pH 7.4 to 1 mg/ml for Virus Immunogens: Influenza H1N1 or H3N2. These immunogens can be used to inject cows.

### Example 3

### Immunization of Cows with H and N Immunogens

Selected Cows (for this application preferably Holstein), approximately 2-3 years old, are injected with the stock “H” or “N” Immunogen. Preferably, four injections are given 1 week apart. Serum samples are collected two weeks after the last initial injection. If boosters are needed, 2 ml dose is given in each booster (every 6 months). Within 4 weeks, cows produced excellent immunoglobulin levels in the plasma.

### Example 4

### Collecting of Plasma

Once it has been determined that the animals have produced a good concentration of the specific immunoglobulins the plasma is harvested from the animal. A series of standard laboratory tests such as specific H1 or N1 ELISA plates can be used to monitor the levels. This is usually 14 to 21 days after the last booster of immunogen or combinations of immunogens. The animals are preferably placed in a clean stall using a head harness. The plasma is preferably collected aseptically using a certified plasmaphoresis machine. The plasma is preferably collected in sterile filter bags. Samples are taken for analysis. The plasma sample is preferably assayed for total protein, total Igg, specific H1 or N1 immunoglobulins, for Johne's, Mycoplasma, BVD (using PCR and direct isolation and for Salmonella and E. coli using direct plate agar assays. The plasma may then be frozen until needed. This material may be further concentrated by passing it through a filter concentrator.

### Example 5

### Development of Material for Gel Swab

To develop material for a gel swap application, 950 ml of sterile water may be measured and poured into a mixing vessel. 100 mg of benzalkonium HCL is preferably added. 10 g USP grade methylcellulose is preferably added while stirring at about 320 rpm. 10 ml USP grade glycerol is preferably added. 5g of Carbopol 974-NP is preferably added to batch while continuing stirring. The batch is preferably stirred for about 1.5 hours at 320 rpm until it is mixed well. 14 ml of NaOH is preferably added and the batch is preferably stirred for 15 minutes. Check pH. The pH should be 7.0±0.05 ph units. If pH is ok 10 ml purified immunoglobulin is preferably added and the batch is stirred for 15 minutes. When the batch is complete, it is preferably chilled at approximately 4 C for at least 2 hours to complete dissolution of the methylcellulose. At this time the gel preparation is complete. 0.5 ml of the gel preparation is applied to the cotton head of each swab. The swab is preferably sealed in a plastic bag until ready to use.

### Example 6

### Testing the Gel Swab

The nasal cavity of a patient may be swabbed with a gel swab. The gel preferably remains in contact with at least a portion of the nasal membrane or the mucous layer on the membrane. This forms a mechanical barrier with the gel and immunoglobulin.

### Example 7

### Development of Material for Aerosol or Spray

One of the key preparations of the immunoglobulin packages according to the inventions is for use in an Aerosol or spray. Specific immunoglobulin packages that are collected from cows immunized with H1N1 and/or H3N2 antigens in equal amounts for a total of 500 mL. Follow the directions in Example 5 to make the solution for the mist. The total amount is preferably 1 L. The mixture is preferably stirred to get a homogenous solution. The material is preferably cooled and stored at 4° C. until used. The mixture is poured into OTC spray bottles using aseptic techniques. The spray bottle preferably includes a measured dose pump. The cap is removed. This material is preferably sprayed directly into the nasal cavity of the patient. With the head held upright, the nozzle is inserted into nostril and the pump is depressed completely 1 or 2 times. The patient is preferably directed to sniff deeply. This application is preferably repeated up to 3 times daily as needed. The nozzle is preferably wiped clean after each use.

### Example 8

### Development of Material for Oral Mist

One of the key preparations of the immunoglobulin packages according to the inventions is for use in an Oral Mist. Specific immunoglobulin packages that are collected from cows immunized with H1N1 and/or H3N2 antigens in equal amounts for a total of 500 mL. Follow the directions in Example 5 to make the solution for the mist. The total amount is preferably 1 L. The mixture is preferably stirred to get a homogenous solution. The material is preferably cooled and stored at 4° C. until used. The mixture is poured into OTC mist bottles using aseptic techniques. The mist bottle preferably includes a measured dose pump. The cap is removed. This material is preferably sprayed directly into the nasal cavity of the patient. With the head held upright, the nozzle is inserted into nostril and the pump is depressed completely 1 or 2 times. The patient is preferably directed to sniff deeply. This application is preferably repeated up to 3 times daily as needed. The nozzle is preferably wiped clean after each use.

### Example 9

### Sample of Animal Testing of Swine

A group of 77 feeder pigs approximately 60 lbs each were tested with material made in Example 4. The animals were given the material as a supplement to the daily rations on days 0, 7, 14 and 21. The average loss due to respiratory complex on this farm was 7.5% and over 30% were medicated during the first 21 days of placement in pens. During the test period of 62 days, all animals were in excellent condition and ahead of schedule with 0% losses and 0% medicated.

### Example 10

### Sample of Animal Testing of Swine

A group of 80 feeder pigs, approximately 50 lbs and considered the runts of the groups, were tested with material made in Example 4 from the immunoglobulin pool. The animals were given the material as mixed in the daily rations on days 0, 7, 14 and 21. The average losses on this farm due to respiratory complex including swine influenza were 5% during the first 21 days and over 30% were medicated. These were the animals that had not done well in the past. This was the average for the farm over the last 5 years. During the test period of 55 days, all animals were in very good condition and ahead of schedule and better than in the past with 1.25% losses and 0% medicated.

The immunoglobulin packages of this invention strongly interferes with binding to the receptors of the target microorganism with the pasal pharyngeal region of the respiratory tract and, on a cumulative basis, thereby prevents the specific targeted virus or cross-reactive virus from colonizing, and multiplying and moving down the respiratory tract and infecting the lower tract including the lungs. Through the vehicle of a simple spray, mist or by a gel coated-swab, the product essentially supplies the host with specific package preparation designed not to cure any disease in the human or animal but merely to dislodge any resident virus and to prevent the attachment of any newly introduced virus in the upper respiratory tract.

The immunoglobulin package has no direct effect on the host itself, is all natural, leaves no undesirable residue in the human or animal. In addition, since the virus is prevented from multiplying, it will over time (for example 21-30 days) disappear through natural degradation from mucus of the host, eliminating the significant potential source of virus to spread human to human. Properly managed, the risk of cross contaminating other humans is lowered and essentially eliminated. Similar applications could be developed for companion animals, swine or poultry as they too have respiratory problems.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A method of producing an immunoglobulin package which acts as viral inhibitor comprising:
identifying at least one colonizing virus;
selecting at least one target immunogen, wherein the at least one target immunogen is selected to contain inhibitors what will bind with the receptors of the at least one colonizing virus;
selecting at least one producing animal;
inoculating the at least one producing animal with the at least one target immunogen according to a predetermined schedule;
waiting a predetermined period of time sufficient to permit the production of immunoglobulin to the at least one target immunogen in the at least one producing animal;
harvesting plasma from the at least one producing animal; and
processing the plasma to create a viral inhibitor.

2. The method of claim 1 wherein the at least one target immunogen is selected from the class consisting of any Influenza group antigens.

3. The method of claim 1 wherein the at least one target immunogen is selected from the class consisting of H5N1.

4. The method of claim 1 wherein the at least one target immunogen is selected from the class consisting of H1N1

5. The method of claim 1 wherein the at least one target immunogen is selected from the class consisting of H3N2

6. The method of claim 1 wherein the producing animal is selected from the group consisting of bovine, sheep, goats, and equine.

7. The method of claim 1 wherein the selecting at least one producing animal step further comprises:
selecting a healthy animal;
profiling the healthy animal's immunoglobulin concentration;
comparing the healthy animal's immunoglobulin concentration to a predetermined minimum immunoglobulin concentration; and .

8. The method of claim 7 wherein the selecting at least one producing animal step further comprises:
selecting a first group of producing animals containing at least two healthy animals; and
selecting a second group of producing animals containing at least two healthy animals.

9. The method of claim 8 wherein said selecting at least one target immunogen step further comprising selecting a first immunogen and a second immunogen.

10. The method of claim 9 wherein said inoculating step further comprises:
inoculating the first group of producing animals with the first target immunogen; and
inoculating the second group of producing animals with the second target immunogen.

11. The method of claim 1 wherein the first target immunogen is selected from the class consisting of H or N influenza antigens and the second target immunogen is selected from the class containing M antigens.

12. The method of claim 11 wherein the first target immunogen and the second target immunogen are shared through the Influenza virus group.

13. The method of claim 10 wherein said processing step further comprises mixing the plasma harvested from the first group with the plasma harvested from the second group.

14. The method of claim 1 wherein the processing step further comprises removing the immunoglobulin-containing contents from said plasma.

15. The method of claim 14 further comprising mixing the immunoglobulin-containing contents of said plasma with a carrier material to form a gel.

16. The method of claim 15 further comprising applying the gel to the cotton head of a swab.

17. The method of claim 1 further comprising mixing the immunoglobulin-containing contents of said plasma with a carrier material to form a spray.

18. The method of claim 1 further comprising mixing the immunoglobulin-containing contents of said plasma with a carrier material to form a mist.

19. A method comprising:
providing an viral inhibitor which contains at least one immunoglobulin package; and
administering the viral inhibitor to at least one human by distributing the viral inhibitor substantially uniformly into the nasal passages thereby preventing the adherence of viral producing immunogens in the upper respiratory tract, wherein said administering occurs on a predetermined administration schedule.

20. The method of claim 19 further comprising administering the viral inhibitor to at least one a human by swabbing the nostrils of the human with the viral inhibitor.

21. The method of claim 20 further comprising administering the viral inhibitor to at least one a human by spraying the viral inhibitor into the nostrils of the human.

22. A method comprising:
providing an viral inhibitor which contains at least one immunoglobulin package; and
administering the viral inhibitor to at least one animal by distributing the viral inhibitor substantially uniformly into the nasal passages thereby preventing the adherence of viral producing immunogens in the upper respiratory tract, wherein said administering occurs on a predetermined administration schedule.

23. The method of claim 22 further comprising administering the viral inhibitor to at least one animal by swabbing the nostrils of the animal with the immunoglobulin packages.

24. The method of claim 22 further comprising administering the viral inhibitor to at least one animal by spraying the viral inhibitor into the nostrils of the animal.
